(19) United States
(12) Patent Application Publication
(10) Pub. No.: US 2007/0219823 A1
(43) Pub. Date: Sep. 20, 2007

(54) Patient monitoring device for remote patient monitoring

(76) Inventor: Adrian F. Warner, Delafield, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/384,034

(22) Filed: Mar. 17, 2006

(51) Int. Cl. G06Q 50/00; G06Q 10/00; A61B 5/00

(52) U.S. Cl. 705/2; 600/300; 600/301

(57) ABSTRACT

A patient monitoring device is disclosed herein for the remote monitoring of a patient. In an embodiment of the present invention, the monitoring device provides the monitoring of patient physiological parameters and supports bidirectional data and communications transmission with a centrally located clinician. In a further embodiment, the monitoring device provides the patient with medication information such as drug schedules and dosage amounts.

### FIELD OF THE INVENTION

The present invention relates to the field of biotelemetry and remote patient care. More specifically, the invention relates to a remote patient monitoring device for monitoring patient physiological parameters, transmitting data and facilitating patient-clinician communications.

### BACKGROUND OF THE INVENTION

The rising cost of health care is of concern to patients and hospital administrators alike. One way to reduce the cost of medical treatment is to reduce the number of days spent in the hospital as there are large fundamental costs associated with each night spent in a hospital bed. As a result, there has been a continued push towards home centered and/or remote off-site patient treatment and/or convalescence. This presents significant challenges for health care providers due to the lack of personal interaction and the lack of a suitably dedicated IT infrastructure. A particular difficulty with home centered or remote off-site treatment is the lack of a patient-care provider interaction which results in the inability of the health care provider to observe the subtle indicators that are relevant to evaluate the current treatment protocol or any adverse reactions to current treatment. Another challenge of remote care is providing 24/7 care to patients that may live alone. Furthermore, remote off-site care presents new challenges in overcoming the distance between the patient and the health care provider upon the detection of an adverse reaction or event.

The field of remote patient monitoring, or biotelemetry, has developed modern technologies and techniques for the remote physiological monitoring of patients. The current monitoring communication infrastructure including, but not limited to, WIFI, cellular, TCP/IP and VOIP platforms or other future developed technologies are suitable to support health care provider-patient communications. These platforms may be used individually or in combination to provide a medical treatment network supporting voice and data transmission. For a remote patient care network to be viable, a patient monitoring device that provides mobile monitoring of patient physiological data as well as providing communication support with a centralized clinician is necessary. Therefore, it is desirable in the field of biotelemetry and remote patient care to provide a patient monitoring device that supports a variety of patient monitoring functionalities as well as providing a remote communications connection to a clinician. It is also desirable that the patient monitoring device be unburdensome to the patient to facilitate its role in mobile patient monitoring as well as provide for contingency in the event of the loss of a connection to a local network.

### SUMMARY OF THE INVENTION

The provision of remote care is facilitated with a cohesive care delivery system that enables the remote monitoring patient vital parameters, medication, and treatment in addition to supporting full bidirectional patient-clinician communication.

The provision of remote patient care is facilitated by the use of a patient monitoring device that provides communication and data transmission via a local communications network to a centralized hospital computer network. The patient monitoring device provides modular physiological monitoring of necessary patient physiological parameters. The specific physiological signals to be monitored being selected by the clinician based upon the patient's specific ailments and/or prescribed treatments. The patient monitoring device also supports bidirectional patient-clinician communications. These communications may be in the form of voice or text communications, or may be the activation of an alarm signal.

In an embodiment of the present invention, the patient monitoring device provides medication management functionalities including a medication and/or treatment schedule with patient treatment alerts, a medication information database, and patient treatment compliance safeguards.

In another embodiment of the present invention, the patient monitoring device may include other functionalities such as GPS for patient location monitoring, individualized treatment files created by the clinician, a patient treatment diary, and/or an automatically recorded patient treatment event log.

In a further embodiment of the present invention, the patient monitoring device comprises a series of patient parameter escalation rules whereby if monitored physiological and/or other data may automatically create a patient escalation and an alarm signal is sent to the clinician via the local network.

In a still further embodiment of the present invention, the patient monitoring device comprises means for buffering and or storing patient physiological parameters and/or events in the event of a communications loss with the local network. The buffering or storage means also allows the patient monitoring device to later transmit this recorded data when a connection to a local network has been reestablished.

Various other features, objects and advantages of the invention will be made apparent from the following description taken together with the drawings.

### DESCRIPTION OF THE DRAWINGS

The drawings illustrate the best mode presently contemplated of carrying out the invention. In the drawings:

FIG. 1 is an embodiment of the patient monitoring device of the present invention;

FIG. 2 is a schematic diagram of an embodiment of the remote patient care network of the present invention; and

FIG. 3 is a schematic diagram of an embodiment of the patient monitoring device of the present invention.

### DETAILED DESCRIPTION OF THE INVENTION

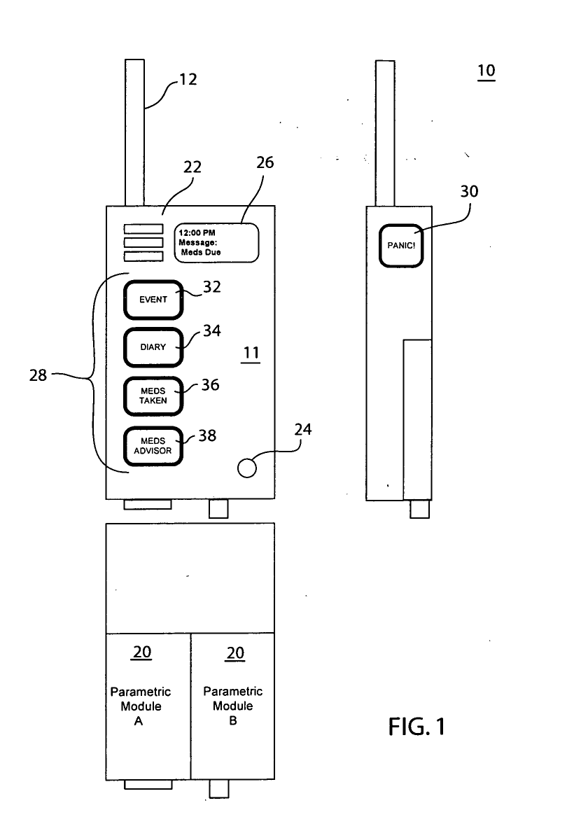

As shown in FIG. 1, patient monitoring device 10 may be a relatively small handheld communications device. The monitoring device 10 comprises a patient interface 11 and a data transmission means 12, pictured in FIG. 1 as an antenna, but may comprise other data transmission means such as a computer cable. Data transmission means 12 connects the patient monitoring device 10 to a local network 14 via WIFI, VOIP, TCP/IP, cellular or other communications platform. Local network 14 provides data transmission to a centralized computer network 16 that may be located at a hospital 18 or some other type of regional medical care facility.

Patient monitoring device 10 comprises a plurality of modular physiological monitoring devices 20. These monitoring devices may be selected by the clinician based upon the specific medical conditions of the patient and the patient's prescribed treatment. Due to the type of care that a patient requires, certain physiological parameters may be more important to monitor than other physiological parameters. These physiological monitoring devices may comprise, but are not limited to, ECG, pulse oximetry, respiration rate, blood pressure, body-temperature, and/or blood glucose monitoring devices. It is also understood in the present invention that the physiological monitoring devices may also comprise stand alone monitoring devices (not shown) that transmit recorded data back to the patient monitoring device 10 via data transmission means 12.

Patient monitoring device 10 also comprises means for facilitating patient-clinician communication. These means may include a speaker 22, a microphone 24, a textual display 26 and a plurality of functionality buttons 28, including an alarm or panic button 30. Vocal patient-clinician communication may be achieved by use of the speaker 22 and microphone 24 and the use of cellular, VOIP, or other communications platforms. This type of communication enables the clinician and the patient to interact, which allows the clinician to obtain so-called “soft” information such as patient responsiveness, reasoning ability, and mood that aids a clinician in evaluating the progression of a treatment. The textual display 26 may similarly be used to support textual communication between the patient and the clinician, or may be used to display preprogrammed patient alerts and/or reminders.

The functionality buttons 28 allow simple patient navigation of the monitoring devices' functionalities. The functionality buttons 28 may comprise an event button 32, diary button 34, a medication log 36, or a medication advisor button 38. The selection of any of these buttons by the patient would prompt the monitoring device 10 to activate that functionality, as will be discussed below. The patient activation of the alarm button 30 would send an alarm transmission to the clinician of the patient-activated alarm condition. This would comprise the fastest form of patient-clinician communication. The clinician may then use the vocal or textual communications means of the patient monitoring device 10 to investigate the purpose for the patient-activated alarm to determine a proper course of action in response to this alarm.

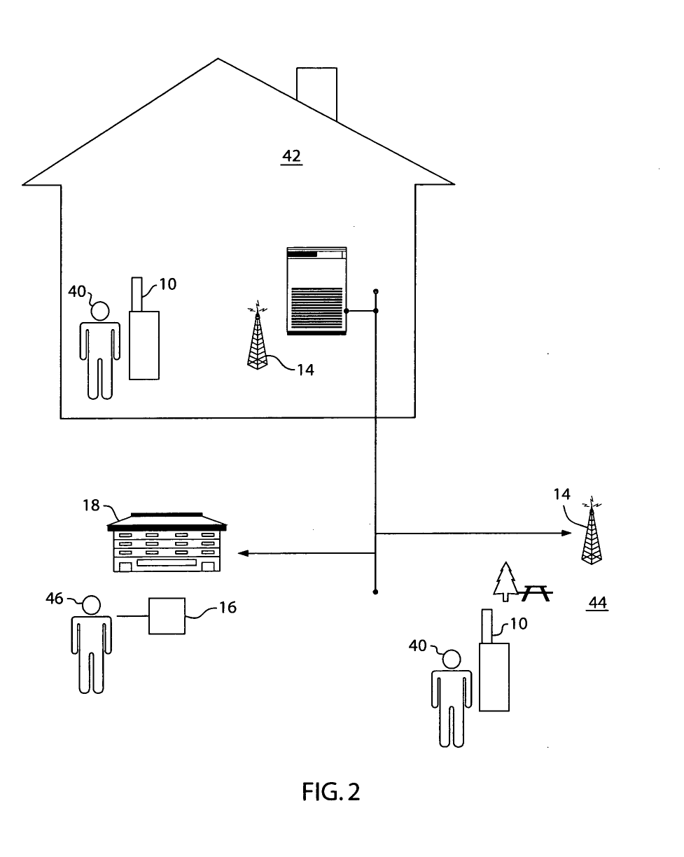

Referring now to FIG. 2 which depicts an embodiment of the remote patient care system using the patient monitoring device 10 of the present invention. A patient 40 has in his possession a properly programmed patient monitoring device 10 of the present invention at some location remote from the primary care hospital 18. The patient 40 may be at a remote location such as his home 42 or some other remote location 44. Wherever the patient 40 is located, the monitoring device 10 searches for and connects to an local network 14. Local network 14 may comprise, but is not limited to, standard communication platforms such as WIFI, cellular, TCP/IP, and VOIP, but any other suitable data transmission platform is contemplated to be within the scope of the present invention. Once a connection with a local network 14 has been established, data and communication may be transmitted to the centralized computer network 16 of a hospital 18 or other regional medical care facility to be reviewed by an attending clinician 46.

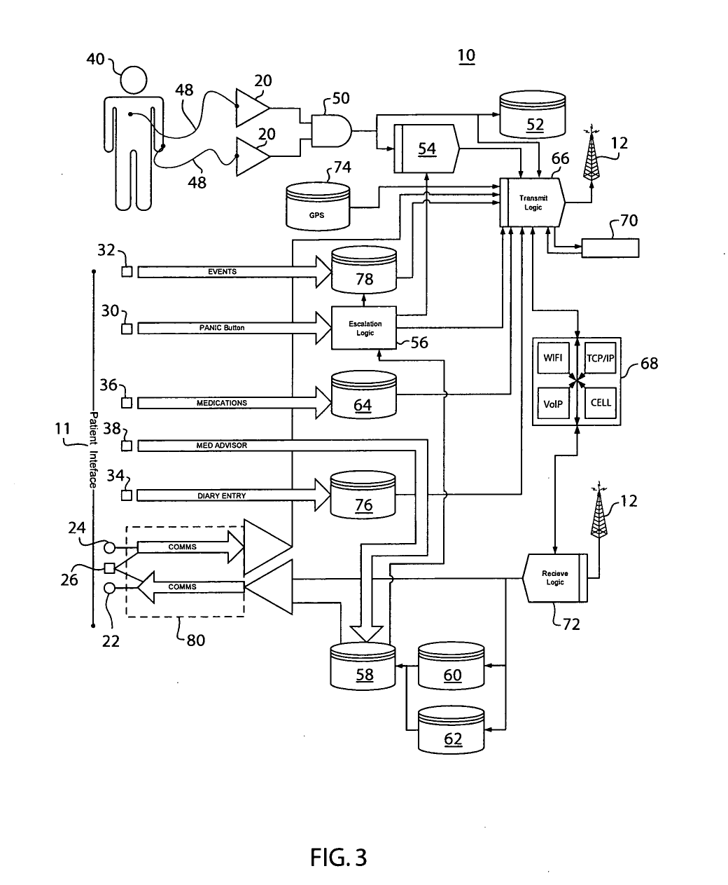

Referring now to FIG. 3, which is a schematic diagram of an embodiment of the patient monitoring device 10 of the present invention. The patient 40 is connected to physiological monitoring devices 20 via leads 48. These measured physiological parameters are collected by a data aggregator 50. This data may be stored in a full disclosure record 52, transmitted to a centralized computer network via the data transmission means 12, or processed with the programmed triggers 54 of the established data escalation logic 56.

Medication management database 58 is comprised of data from the prescription master file 60 and the medication information database 62. The prescription master file is a data file that is created by the clinician upon the establishment of the patient's remote care treatment regimen. The prescription master file 60 may comprise both institutionally created standard practices, treatments or procedures, as well as additional clinician specified treatments and procedures to provide an individualized patient prescription file. This file will also comprise patient medication prescriptions and medication schedules for the patient's specified treatment. The medication information database 62 provides a resource of additional information regarding the patient's diagnosis, treatment, and medications. This information may include, but is not limited to, medication names, manufacturers, dosage amounts, dosage schedules, and common side effects.

In response to monitored patient physiological parameters, or based on communications between the patient and clinician, the clinician may transmit new instructions or prescription information to the master file 60 via transmission means 12. This allows the clinician to modify the patient's treatment remotely, reducing the number of face-to-face hospital visits required for the patient to attend.

The medication management database 58 allows the monitoring device 10 to remind the patient 40 of the patient's medication schedule through both audio and textual medication reminders and prompts. These prompts would include the name of the drug, the dosage amount, and any additional instructions and/or warnings, such as the instruction to take the medication with food, or the warning to not operate heavy machinery after taking this medication.

The monitoring device 10 also promotes patient compliance with the medication schedule by notifying the clinician 46 of the patient medication alerts so that the clinician can contact the patient using the communications features of the present invention to check up on or to verify that the patient has taken the scheduled medication.

In an alternative embodiment of the present invention, the monitoring device 10 of the present invention would prompt the patient 40 to use an infrared or similar scanner to scan a barcode that is located on the medication bottle to verify that the patient has taken the proper medication.

In a still further embodiment of the present invention, the medication management database 58 includes escalation logic for the detection of the drug desired affect. For example, in an embodiment of the present invention, a patient prescription master file 60 may include the directive that the patient take a dosage of aspirin daily. The medication management database 58 may include the additional escalation logic 56 of looking for a decrease in patient blood pressure in the time period after which the patient was supposed to have taken the aspirin dosage. Presumably, if the patient has taken his aspirin dosage, the escalation logic will see that there is a decrease in patient blood pressure and as such, verify that the patient has complied with his treatment regimen. If the escalation logic 56 does not detect a decrease in patient blood pressure, then this could trigger an alarm to the clinician 46 that the patient 40 has not taken his prescribed dosage of aspirin.

In addition to the compliance features of the medication management database 58, the patient 40 may also access the medication management database 58 for medication and/or treatment information. In an embodiment of the present invention, the patient may press the meds taken button 36 of the patient interface 11 to access the patient's medication record 64, which would display the medication history of the patient over the course of his treatment. Alternatively, in an embodiment of the present invention, the patient 40 may press the medication advisor button 38 to access information from the medication information database 62 to find out additional information about the medications that the patient is currently taking. These features provide limited instantaneous feedback that could answer a variety of patient questions, thus improving the quality of the remote care that is provided to the patient.

In an embodiment of the present invention, the escalation logic 56 may include a variety of institutionally established and/or clinician established alarm parameters. These escalation logic parameters are used to analyze the recorded patient physiological parameters and to set programmed triggers 54 for automatic alarms to be sent to the clinician 46 via the local network 14. These programmed triggers 54 may include specified ranges for heart rate, blood pressure, temperature, blood glucose, or any other physiological parameter that the patient monitoring device 10 may be monitoring the patient for. If the detected parameter is outside of this specified range, an automatic alert signal is generated and sent to the clinician 46. Alternatively, the programmed triggers 54 may include more complex physiological data analysis such as arrhythmia detection or boolean logic statements combining ranges for multiple physiological parameters, producing an automatic alarm if these triggering conditions are met.

In an embodiment of the present invention, bidirectional patient-clinician communication is achieved by voice communications and/or textual communications systems 80. Patient voice communications are received by microphone 24 and sent to transmit logic 66 to be sent to the clinician. Similarly, clinician voice transmissions are received by receive logic 72 and sent to the speaker 22 via communication system 80. Alternatively, the patient monitoring device 10 may include textual communications support. Textual display 26 would facilitate this bidirectional textual communications by displaying messages from the clinician and allowing the patient to enter textual communications to be sent back to the clinician.

The data and communications transmissions are facilitated between the patient monitoring device 10 and the local network 14 by data transmission means 12 which may be comprised as an antenna on patient monitoring device 10 and such data transmission means 12 may employ a variety of communications platforms including, but not limited to, WIFI, cellular, TCP/IP, or VOIP platforms. Before data is transmitted from the monitoring device 10, to the centralized computer network 16, the data is filtered through transmit logic 66. The transmit logic 66 verifies the existence of available communications platforms to the data transmission means 12 for data transmission. The transmit logic 66 uses optimization logic 68 to select an optimal available communications platform. Optimization logic 68 may include such factors as cost of the connection, the bandwidth of the connection, or the strength of the connection signal. Once a communications platform has been selected, the transmit logic 66 connects to the local network 14 utilizing this communications platform and transmits the data from the monitoring device 10 to the centralized computer network 16.

Alternatively, if the optimization logic determines that there is no available communications platform, then the transmit logic stores the data to be transmitted in a buffer or storage means 70 where the data remains until a local network 14 with an available communications platform is present. At that point, the transmit logic 66 will connect to that local network 14 and transmit the data that has been stored in the buffer or storage means 70. Similarly, receive logic 72 uses optimization logic 68 to determine the available and desirable local networks 14 from which to receive data.

In an alternative embodiment of the present invention, the monitoring device 10 includes a global positioning system (GPS) 74 or other similar location tracking component. This could provide the clinician 46 with valuable location information about the patient 40 in the event of an emergency or alarm situation. Since the patient is being monitored remotely, if there is an emergency situation, then the precise location of the patient may not be known, thereby making the delivery of emergency care to the patient difficult. GPS 74 functionality could help to alleviate this problem with the delivery of remote patient care.

Still further embodiments of the present invention may include a full disclosure record 52 that works as a silent recorder of all the detected patient physiological parameters, thus keeping a complete record of the parameters measured. An alternative embodiment of the present invention may include a diary 76 whereby the patient 40 may record his thoughts verbally by pressing the diary button 34 at the patient interface 11. This would record an ongoing record of additional patient data including patient questions and/or concerns that would be transmitted back to the centralized computer network 16 to be accessible to the clinician 46 to aid in further patient treatment.

In a further embodiment of the present invention, the patient monitoring device 10 may include an event record 78 whereby any events during the patient's treatment, such as the taking of medication, the updating of a patient's prescription, or the activation of any alarm signals is recorded. This record may be accessed by the patient via the event button 32 and is also transmitted to the centralized computer network 16 to be accessible to the clinician 46 to aid in the remote monitoring of the patient 40.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements of insubstantial differences from the literal language of the claims.

Various alternatives and embodiment are contemplated as being within the scope of the following claims, particularly pointing out and distinctly claiming the subject matter regarded as the invention.

I claim:

1. An integrated medical device for the remote monitoring of a patient by a clinician on a centralized computer network where a clinician receives medical data from the patient and provides technical and communicative assistance to the patient, the device comprising:
a local network wherein data is provided from a discrete patient location to the centralized computer network; and
diagnostic technology for monitoring the patient's medical condition;
a data transmission means for sending and receiving data and communication signals to the receiving station; and
a programmable functionality to provide assistance to the patient;
wherein the medical device provides convalescent support to the patient via the transmission of medical data to the clinician and remote communication with the clinician when the monitor is connected to a local network.

2. The integrated medical device of claim 1 wherein the programmable functionality comprises the management of the patient's medicine schedule and dosage.

3. The integrated medical device of claim 2 wherein the patient monitor provides a reminder to the patient to take a medication.

4. The integrated medical device of claim 3 wherein the patient monitor provides data input means whereby the patient may access drug information, dosage schedule, and drug instructions.

5. The integrated medical device of claim 1 wherein the programmable functionality comprises a patient diary.

6. The integrated medical device of claim 1 wherein the patient monitor further comprises data storage, the data storage comprising medication information, stored patient physiological data, and an event log.

7. The integrated medical device of claim 1 further comprising optimization logic for selecting the most desirable available transmitter connection for data transmission.

8. The integrated medical device of claim 7 wherein the programmable functionality remains operational despite a lack of a transmitter connection with the centralized computer network.

9. The device of claim 8 wherein the monitor records any monitored data, the data being sent to the centralized computer network upon the establishment of a transmitter connection with the centralized computer network.

10. A method of remotely treating a patient from a centralized location via the transmission of data and communication from the centralized location to a remote device in the possession of the patient, the method comprising the steps of:
transducing physiological data via a physiological monitoring means of the remote device;
establishing a communication link between the clinician and the patient via the remote device via one of a plurality of possible communication modes;
sending the automatic alarm conditions to the centralized location; and
providing bidirectional communication support between the patient and the clinician via the remote device.

11. The method of remotely treating a patient according to claim 10, wherein the plurality of communication modes comprise WIFI, cellular, TCP/IP, and VOIP.

12. The method according to claim 11, wherein the communication mode is selected based upon the optimization of available communication modes.

13. The method according to claim 12, wherein the remote device functionalities are available to the patient independent of the ability to establish a communication link.

14. The method according to claim 10 further comprising the step of providing patient medication information.

15. The method of remotely treating a patient according to claim 9 wherein the communication link comprises vocal, textual, and alarm condition communications.

16. A patient monitoring device for the management of a patient's medication and tracking of a patient's physiological condition while the patient is at a location remote from a hospital, the monitoring device comprising:
a patient monitoring means for the transduction of the patient's physiological parameters;
a transmission means to connect the monitoring device to a centralized computer network whereby data may be sent between the centralized computer network and the monitoring device;
digital data storage for storing drug information, patient dosage information, and patient medication schedule information;
a patient notification means to alert the patient to a reminder, information or a communication;
a data input means for the patient to enter medical or communication information; and
escalation logic with a plurality of programmed triggers for the activation of an automatic alarm based upon the patient's individual physiological parameters;
wherein the patient receives a reminder via the notification means to take a prescribed medication at a particular time as according to the patient's medication schedule information.

17. The device of claim 16 wherein the patient notification includes vocal and visual instructions for medication delivery.

18. The device of claim 16 wherein the patient monitoring means further comprises a plurality of modular physiological data monitoring devices.

19. The device of claim 18 wherein the diagnostic technology comprises stand alone physiological data monitoring devices that communicate to the patient monitoring device via the data transmission means.

20. The device of claim 18 wherein the plurality of modular physiological data monitoring devices is selected form the group comprising: ECG, blood pressure, respiration rate, pulse oximetry, blood glucose, temperature, and patient position.
